(19) United States
(12) Patent Application Publication
(10) Pub. No.: US 2020/0170833 A1
(43) Pub. Date: Jun. 4, 2020

(54) VERSATILE THERAPEUTIC EYE MASK

(71) Applicant: Shanghai GK Industrial Co.,Ltd., Shanghai (CN)

(72) Inventor: Jiangang DUAN, Shanghai (CN)

(73) Assignee: Shanghai GK Industrial Co.,Ltd., Shanghai (CN)

(21) Appl. No.: 16/231,975

(22) Filed: Dec. 25, 2018

(30) Foreign Application Priority Data
Dec. 4, 2018 (CN) 201822021996.0

(51) Int. Cl. A61F 7/00; A61M 21/00

(52) U.S. Cl. CPC A61F 7/007; A61M 21/00; A61M 2021/0044; A61M 2021/0022; A61B 5/4806; A61F 2007/0004; A61F 2007/0078; A61F 2007/0094; A61F 2007/0096; A61M 2021/0083

(57) ABSTRACT

A versatile therapeutic eye mask includes a mask body and a strap connected at two ends of the mask body. A heating layer, a heat storage and conduction layer, and a supersoft flannel contact layer are sequentially disposed in the mask body from inside to outside. The heating layer includes a heating sheet. The heating sheet is connected to a rechargeable battery. The rechargeable battery is connected to a controller. A first side of the heat storage and conduction layer is in contact with the heating layer. A second side of the heat storage and conduction layer is in contact with the supersoft flannel contact layer. A patch temperature sensor is bonded on the first side. The patch temperature sensor is connected to the controller. The supersoft flannel contact layer is movably bonded on the mask body.

### CROSS REFERENCE TO RELATED APPLICATIONS

This application is based upon and claims priority to Chinese Patent Application No. 201822021996.0, filed on Dec. 4, 2018, the entire contents of which are incorporated herein by reference.

### TECHNICAL FIELD

The present invention relates to the technical field of eye masks, and in particular, to a versatile therapeutic eye mask.

### BACKGROUND

Irregular lifestyle and lack of sleep at night that are caused by various reasons leave modern people easily susceptible to problems such as dark circles, eye dryness, eye irritation, blepharitis, and styes. Such problems easily cause epigenetic abnormalities and endocrine disorders in the long run and severely affect physical health.

People often choose to take an afternoon nap to make up for the lack of sleep at night. However, limited conditions during an afternoon nap make it difficult to create an environment similar to a sleep environment at night. People then choose to wear eye masks to simulate a sleep environment at night. At present, most commercially available eye masks are used to block light and eliminate interference to create an environment suitable for sleeping and are suitable for daytime use and outdoor use. However, such eye masks lack versatility and cannot alleviate the problems such as dark circles, eye dryness, eye irritation, blepharitis, and styes.

### SUMMARY

The objective of the present invention is to alleviate problems such as dark circles, eye dryness, eye irritation, blepharitis, and styes of people due to lack of sleep at night. The present invention provides a versatile therapeutic eye mask.

To achieve the foregoing objective, the technical solution of the present invention is as follows:

A versatile therapeutic eye mask includes a mask body and a strap connected at two ends of the mask body, where a heating layer, a heat storage and conduction layer, and a supersoft flannel contact layer are sequentially disposed in the mask body from inside to outside, the heating layer includes a heating sheet, the heating sheet is connected to a rechargeable battery, and the rechargeable battery is connected to a controller;

one side of the heat storage and conduction layer is in contact with the heating layer, the other side of the heat storage and conduction layer is in contact with the supersoft flannel contact layer, a patch temperature sensor is bonded on the side, and the patch temperature sensor is connected to the controller; and the supersoft flannel contact layer is movably bonded on the mask body.

As an improvement, several through holes are provided on a lateral side of the mask body.

As an improvement, a sleep monitoring sensor is disposed on the supersoft flannel contact layer, and the sleep monitoring sensor is connected to the controller.

As an improvement, a touch display screen, a battery charging interface, a vibrator, and an adjustment button are further disposed on the mask body, the touch display screen, the vibrator, and the adjustment button are connected to the controller, and the battery charging interface is connected to the rechargeable battery.

As an improvement, several LED lamp strips are disposed inside the mask body along an inner side edge of the mask body, and the LED lamp strips are connected to the controller.

As an improvement, the controller is further connected to a Bluetooth communications device.

As an improvement, several air holes are provided in the supersoft flannel contact layer, and a connecting line of centers of the air holes in the supersoft flannel contact layer forms an ellipse.

Beneficial effects of the present invention are as follows:

1. The heating layer, the heat storage and conduction layer, and the supersoft flannel contact layer are used to transfer heat to eyes to accelerate blood flow and help alleviate the problems such as dark circles, eye dryness, eye irritation, blepharitis, and styes of people due to the lack of sleep at night.

2. The sleep monitoring sensor is used to implement monitoring of sleep time and provide a vibration wake-up function and a light wake-up function.

3. The several air holes are provided in the supersoft flannel contact layer, and the connecting line of the centers of the air holes in the supersoft flannel contact layer forms an ellipse, so that heat is mainly applied around eyes through the air holes.

### BRIEF DESCRIPTION OF THE DRAWINGS

FIG. 1 is a schematic structural view of a heating layer;

FIG. 2 is a schematic structural view of a heat storage and conduction layer;

FIG. 3 is a schematic structural view of a supersoft flannel contact layer;

FIG. 4 is a front view of a versatile therapeutic eye mask;

FIG. 5 is a first overall schematic structural view of a versatile therapeutic eye mask;

FIG. 6 is a second overall schematic structural view of a versatile therapeutic eye mask; and

FIG. 7 is a schematic view of a connection relationship between a controller and other components.

Where: 1. mask body; 2. heating layer; 3. rechargeable battery; 4. heat storage and conduction layer; 5. patch temperature sensor; 51. first patch temperature sensor; 52. second patch temperature sensor; 6. supersoft flannel contact layer; 7. air hole; 8. sleep monitoring sensor; 9. battery charging interface; 10. vibrator; 101. first vibrator; 102. second vibrator; 11. touch display screen; 12. adjustment button; 13. strap; 14. through hole; 15. controller; and 16. LED lamp strip.

### DETAILED DESCRIPTION

The present invention is further described below in detail with reference to specific implementations.

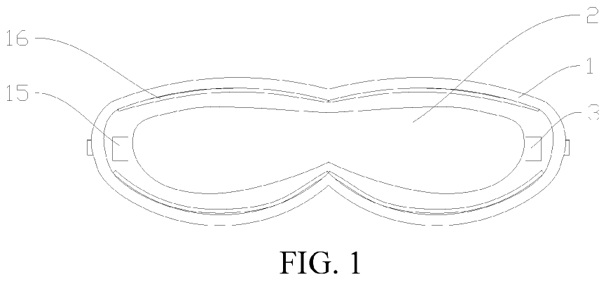

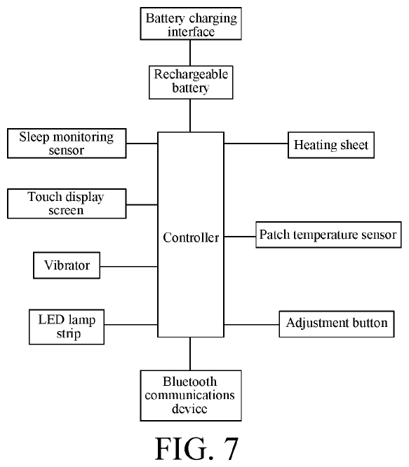

Referring to FIG. 1, a versatile therapeutic eye mask includes a mask body 1 and a strap 13 connected at two ends of the mask body 1. In this embodiment, the mask body 1 is made of a soft fabric material. A heating layer 2, a heat storage and conduction layer 4, and a supersoft flannel contact layer 6 are sequentially disposed in the mask body 1 from inside to outside. The heating layer 2 includes a heating sheet. The heating sheet is made of metal fiber filaments or carbon fiber or graphene, but is not limited to the foregoing materials. The heating sheet is connected to a rechargeable battery 3. The rechargeable battery 3 is connected to a controller 15, as shown in FIG. 7. The rechargeable battery 3 is a lithium battery that has a small size and a light weight. The rechargeable battery 3 may be disposed inside or outside the versatile therapeutic eye mask.

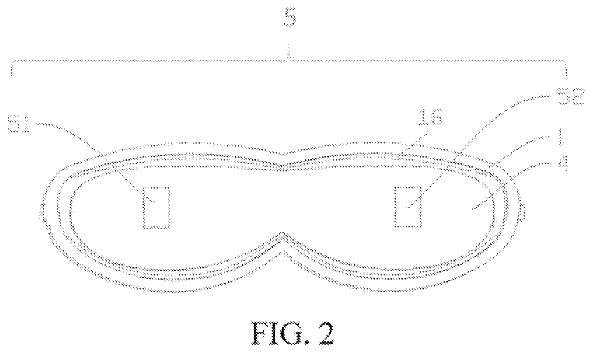

Referring to FIG. 2, one side of the heat storage and conduction layer 4 is in contact with the heating layer 2. The other side of the heat storage and conduction layer 4 is in contact with the supersoft flannel contact layer 6. A patch temperature sensor 5 is bonded on the side. The patch temperature sensor 5 is connected to the controller 15, as shown in FIG. 7. The controller 15 is further connected to a Bluetooth communications device.

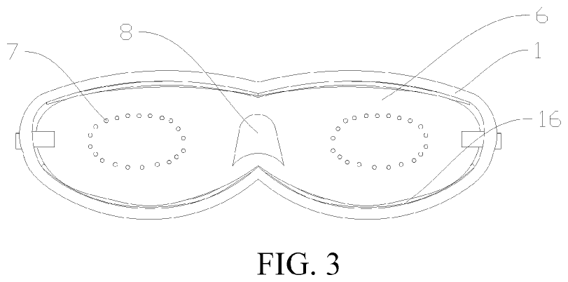

Referring to FIG. 3, the supersoft flannel contact layer 6 is movably bonded on the mask body 1. Several air holes 7 are provided in the supersoft flannel contact layer 6. A connecting line of centers of the air holes 7 in the supersoft flannel contact layer 6 forms an ellipse. A sleep monitoring sensor 8 is disposed on the supersoft flannel contact layer 6. The sleep monitoring sensor 8 is connected to the controller 15, as shown in FIG. 7.

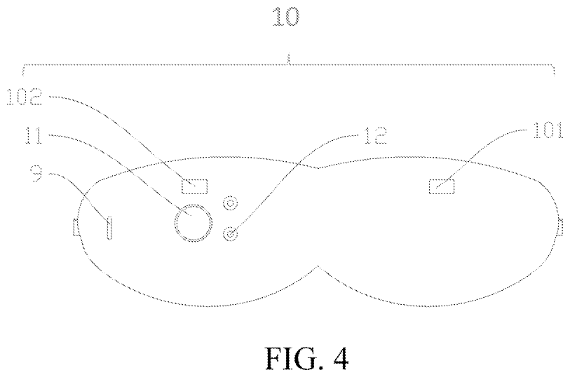

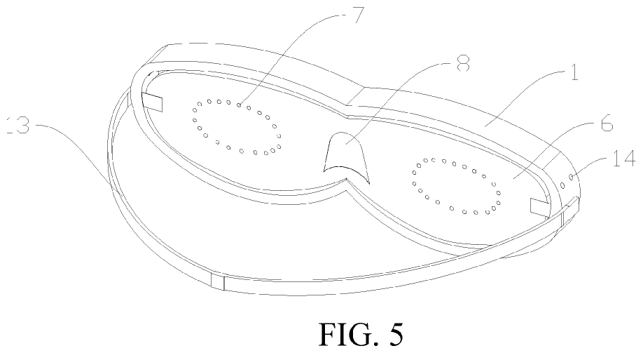

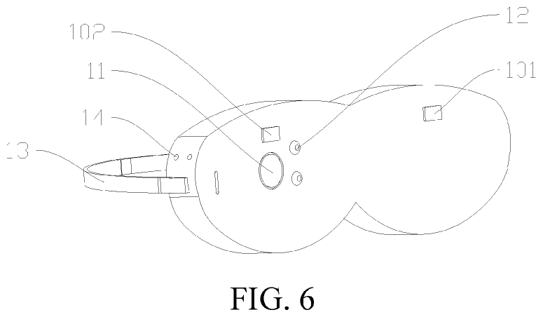

Referring to FIG. 4, FIG. 5, and FIG. 6, several through holes 14 are provided on a lateral side of the mask body 1. A touch display screen 11, a battery charging interface 9, a vibrator 10, and an adjustment button 12 are further disposed on the mask body 1. The touch display screen 11, the vibrator 10, and the adjustment button 12 are connected to the controller 15, as shown in FIG. 7. The battery charging interface 9 is connected to the rechargeable battery 3. Several LED lamp strips 16 are disposed inside the mask body 1 along an inner side edge of the mask body 1. The LED lamp strips 16 are connected to the controller 15. In this embodiment, the battery charging interface 9 is a micro USB interface.

The heating layer 2, the heat storage and conduction layer 4, and the supersoft flannel contact layer 6 are used to transfer heat to eyes to accelerate blood flow and help alleviate problems such as dark circles, eye dryness, eye irritation, blepharitis, and styes of people due to lack of sleep at night. The sleep monitoring sensor 8 is used to implement monitoring of sleep time and provide a vibration wake-up function and a light wake-up function. The several air holes 7 are provided in the supersoft flannel contact layer 6, and the connecting line of the centers of the air holes 7 in the supersoft flannel contact layer 6 forms an ellipse, so that heat is mainly applied around eyes through the air holes 7.

When the eye mask is used for heat application, the touch display screen 11, the adjustment button 12, and the controller 15 are used to set a heating temperature and heat application duration. A heating temperature that reaches the set temperature flashes on the touch display screen 11. At this time, the eye mask is put on, the strap 13 is used to make an adjustment to appropriate tightness, and the heat application is started. During the heat application, the patch temperature sensor 5 senses a current temperature of the heat application in real time. The controller 15 controls working of the heating sheet to keep the set heating temperature. Heat from the heating sheet sequentially passes through the heat storage and conduction layer 4, the supersoft flannel contact layer 6, and the air holes 7 in the supersoft flannel contact layer 6 to be applied to eyes. After the heat application ends, the LED lamp strips 16 flash and the vibrator 10 vibrates. Excessive heat dissipates through the through holes 14 in the mask body 1. In this embodiment, two patch temperature sensors 5, namely, a first patch temperature sensor 51 and a second patch temperature sensor 52, are separately bonded at two ends of the heat storage and conduction layer 4. Two vibrators 10, namely, a first vibrator 101 and a second vibrator 102, are mounted at the two ends of the mask body 1.

When the eye mask is used to create a desirable sleep environment, the touch display screen 11, the adjustment button 12, and the controller 15 are used to set a fixed sleep time value. The sleep monitoring sensor 8 detects depth sleep time of a wearer. The controller 15 performs conversion on data of the depth sleep time and displays the data on the touch display screen 11. In addition, the controller 15 may also send the data to a mobile phone terminal by using the Bluetooth communications device for viewing.

When sleep time of a user reaches the set fixed sleep time value, the controller 15 controls the LED lamp strips 16 to emit soft ambient light and the vibrator 10 vibrates to wake up the user.

When the eye mask is used to perform heat application and create a desirable sleep environment, the touch display screen 11, the adjustment button 12, and the controller 15 may be used to set a heating temperature, heat application duration, and a fixed sleep time value. The LED lamp strips 16 and the vibrator 10 do not perform any action after the heat application ends and perform corresponding actions after sleep ends.

When the rechargeable battery 3 runs out, the rechargeable battery 3 may be charged by using the battery charging interface 9 disposed on the mask body 1.

Finally, it should be noted that the foregoing embodiments are merely provided for describing the technical solutions of the present invention, but are not intended to limit the present invention. It should be understood by persons of ordinary skill in the art that although the present invention has been described in detail with reference to the preferred embodiments, modifications can be made to the specific implementations of the present invention or equivalent replacements can be made to some technical features without departing from the spirit of the technical solutions of the present invention. These modifications and equivalent replacements shall all fall within the scope of the technical solutions that the present invention seeks to protect.

What is claimed is:

1. A versatile therapeutic eye mask, comprising:
a mask body, and
a strap connected at two ends of the mask body;
wherein
a heating layer, a heat storage and conduction layer, and a supersoft flannel contact layer are sequentially disposed in the mask body from inside to outside, the heating layer comprises a heating sheet, the heating sheet is connected to a rechargeable battery, and the rechargeable battery is connected to a controller;
a first side of the heat storage and conduction layer is in contact with the heating layer, a second side of the heat storage and conduction layer is in contact with the supersoft flannel contact layer, a patch temperature sensor is bonded on the first side of the heat storage and conduction layer, and the patch temperature sensor is connected to the controller; and
the supersoft flannel contact layer is movably bonded on the mask body.

2. The versatile therapeutic eye mask according to claim 1, wherein a plurality of through holes are provided on a lateral side of the mask body.

3. The versatile therapeutic eye mask according to claim 1, further comprising a sleep monitoring sensor disposed on the supersoft flannel contact layer; wherein the sleep monitoring sensor is connected to the controller.

4. The versatile therapeutic eye mask according to claim 1, further comprising a touch display screen, a battery charging interface, a vibrator, and an adjustment button disposed on the mask body; wherein the touch display screen, the vibrator, and the adjustment button are connected to the controller; and the battery charging interface is connected to the rechargeable battery.

5. The versatile therapeutic eye mask according to claim 1, further comprising a plurality of LED lamp strips disposed inside the mask body along an inner side edge of the mask body; wherein the plurality of LED lamp strips are connected to the controller.

6. The versatile therapeutic eye mask according to claim 1, wherein the controller is connected to a Bluetooth communications device.

7. The versatile therapeutic eye mask according to claim 1, further comprising a plurality of air holes provided in the supersoft flannel contact layer; wherein a connecting line of a plurality of centers of the plurality of air holes in the supersoft flannel contact layer forms an ellipse.
